(19) United States
(12) Patent Application Publication
(10) Pub. No.: US 2008/0264414 A1
(43) Pub. Date: Oct. 30, 2008

(54) METHOD AND SYSTEM FOR CONSERVING ANESTHESIA, HEAT AND MOISTURE

(75) Inventors: Grant M. McNeilly, Monona, WI (US); Robert Q. Tham, Middleton, WI (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/741,406

(22) Filed: Apr. 27, 2007

(51) Int. Cl. A61M 16/10

(52) U.S. Cl. 128/203.12; 128/205.27

(57) ABSTRACT

A method for absorbing an anesthetic, heat and moisture in an anesthesia system is disclosed herein. The method includes providing a first material configured to absorb an anesthetic transferred from a patient. The method also includes providing a second material configured to absorb heat and moisture transferred from the patient. The method also includes combining the first material with the second material in order to produce a single composite material adapted to generally simultaneously absorb the anesthetic, heat and moisture.

### FIELD OF THE INVENTION

This disclosure relates generally to a method and system for conserving anesthesia, heat and moisture.

### BACKGROUND OF THE INVENTION

Anesthesia may be administered to a patient in the form of a gas for purposes such as blocking the conscious perception of pain, producing unconsciousness, preventing memory formation, and/or preventing unwanted movement. The administered anesthetic agent is inhaled into the patient's lungs. Thereafter, the patient absorbs a fraction of the administered anesthetic agent and exhales the remainder. One problem is that the anesthetic agent is expensive and it is therefore costly to waste the exhaled anesthetic agent. Additionally, the exhaled anesthetic agent can pose a health risk to nearby personnel and may be environmentally unsafe.

In addition to exhaling anesthetic agent, the patent also expels heat and moisture. This loss of heat and moisture can lower the patient's body temperature and can dry out the patient's respiratory tract. These effects can cause discomfort and can increase the potential for complications or other health concerns.

### BRIEF DESCRIPTION OF THE INVENTION

The above-mentioned shortcomings, disadvantages and problems are addressed herein which will be understood by reading and understanding the following specification.

In an embodiment, a method for conserving anesthetic, heat and moisture in an anesthesia system includes providing a first material configured to absorb an anesthetic transferred from a patient. The method also includes providing a second material configured to absorb heat and moisture transferred from the patient. The method also includes combining the first material with the second material in order to produce a single composite material adapted to generally simultaneously absorb the anesthetic, heat and moisture.

In another embodiment, an absorber for an anesthesia system includes a first material configured to absorb an anesthetic transferred from a patient. The absorber also includes a second material engaged with the first material. The second material is configured to absorb heat and moisture transferred from the patient. The first material and the second material are combined in a manner configured to provide generally simultaneous absorption of the anesthetic, heat and moisture.

In another embodiment, an anesthesia system includes a vaporizer configured to convert a liquid anesthetic agent into a gaseous anesthetic agent, and a breathing tube in communication with the vaporizer. The breathing tube is configured to transfer the gaseous anesthetic agent to a patient. The anesthesia system also includes an absorber in communication with the patient. The absorber is disposed remotely relative to the vaporizer. The absorber includes a first material comprising carbon. The first material is configured to absorb the gaseous anesthetic agent transferred from the patient. The absorber also includes a second material engaged with said first material. The second material is configured to absorb heat and moisture transferred from the patient. The first material and the second material are combined in a manner configured to provide generally simultaneous absorption of the anesthetic, heat and moisture.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a anesthesia system including an absorber in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

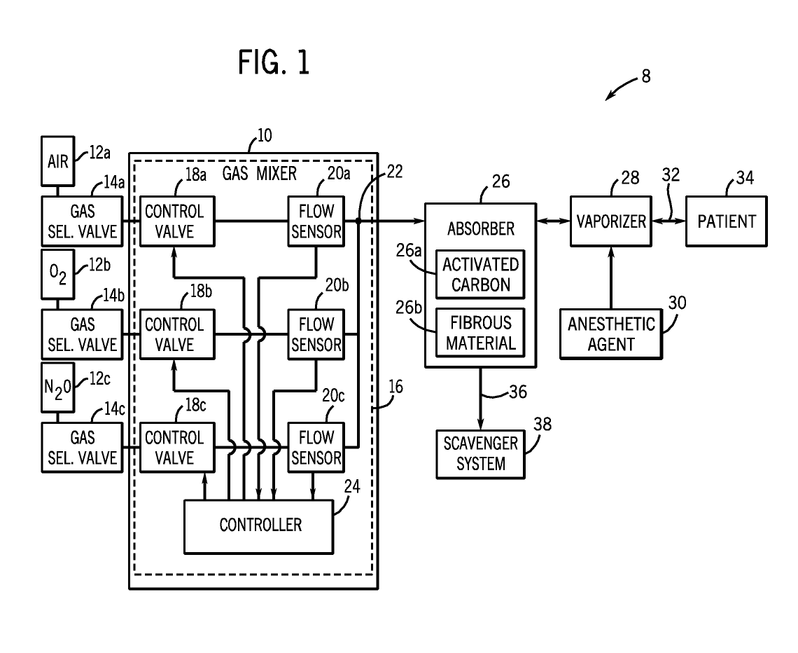

Referring to FIG. 1, an anesthesia system 8 is schematically depicted in accordance with one embodiment. The anesthesia system 8 includes an anesthesia machine 10, a plurality of gas storage devices 12a, 12b and 12c, an absorber 26, a vaporizer 28, and a scavenger system 38. The anesthesia machine 10 is shown for illustrative purposes and it should be appreciated that other types of anesthesia machines may alternately be implemented. In a typical hospital environment, the gas storage devices 12a, 12b and 12c are centrally located storage tanks configured to supply medical gas to multiple anesthesia machines and multiple hospital rooms. The storage tanks are generally pressurized to facilitate the transfer of the medical gas to the anesthesia machine 10.

The gas storage devices 12a, 12b and 12c will hereinafter be described as including an air tank 12a, an oxygen (O₂) tank 12b, and a nitrous oxide (N₂O) tank 12c, respectively, however it should be appreciated that other storage devices and other types of gas may alternatively be implemented. The gas storage tanks 12a, 12b and 12c are each connected to one of the gas selector valves 14a, 14b, and 14c, respectively. The gas selector valves 14a, 14b and 14c may be implemented to shut off the flow of medical gas from the storage tanks 12a, 12b and 12c when the anesthesia machine 10 is not operational. When one of the gas selector valves 14a, 14b and 14c is opened, gas from a respective storage tank 12a, 12b and 12c is transferred under pressure to the anesthesia machine 10.

The anesthesia machine 10 includes a gas mixer 16 adapted to receive medical gas from the storage tanks 12a, 12b and 12c. The gas mixer 16 includes a plurality of control valves 18a, 18b and 18c that are respectively connected to one of the gas selector valves 14a, 14b and 14c. The gas mixer 16 also includes a plurality of flow sensors 20a, 20b and 20c that are each disposed downstream from a respective control valve 18a, 18b, and 18c. After passing through one of the control valves 18a, 18b and 18c, and passing by one of the flow sensors 20a, 20b and 20c, the individual gasses (i.e., air, O₂ and N₂O) are combined to form a mixed gas at the mixed gas outlet 22.

The control valves 18a, 18b and 18c and the flow sensors 20a, 20b and 20c are each connected to a controller 24. The controller 24 is configured to operate the control valves 18a, 18b and 18c in a response to gas flow rate feedback from the sensors 20a, 20b and 20c. Accordingly, the controller 24 can be implemented to maintain a selectable flow rate for each gas (i.e., air, O₂ and N₂O) such that the mixed gas at the mixed gas outlet 22 comprises a selectable ratio of air, O₂ and N₂O. The mixed gas flows through an absorber 26 to a vaporizer 28 where an anesthetic agent 30 is vaporized and added to the mixed gas from the mixed gas outlet 22. The anesthetic agent 30 and mixed gas combination passes through a breathing tube 32 and is delivered to the patient 34. Although the vaporizer 28 and anesthetic agent 30 are schematically depicted as being separate components of the anesthesia system 8, it should be appreciated that one or both of these components may alternatively be incorporated into the design of the anesthesia machine 10.

A fraction of the anesthetic agent 30 administered to the patient 34 is absorbed into the patient's blood stream, and the remainder is expelled as the patient 34 exhales. The exhaled anesthetic agent 30 is transferred via the breathing tube 32 back to the absorber 26. A portion of the exhaled anesthetic agent 30 can be collected by the absorber 26 and re-directed back to the patient 34 during a subsequent inhalation. The absorber 26 is similarly configured to collect and re-direct exhaled heat and moisture as will be described in detail hereinafter. Advantageously, the absorber 26 configured in the manner described recycles anesthetic agent 30 as a cost savings measure, and also limits the patient's loss of heat and moisture in order to maintain patient comfort and to minimize the risk of complications. The uncollected exhaled anesthetic agent 28 is transferred through the scavenger tube 36, into the scavenger system 38 and is released into the atmosphere where it becomes diluted with outside air to the extent that it is no longer dangerous.

According to one embodiment, the absorber 26 comprises activated carbon 26a combined with a fibrous material 26b. The activated carbon 26a is adapted to absorb anesthetic agent 30 exhaled from the patient 34. The fibrous material 26b is adapted to absorb moisture exhaled from the patient 34. The process of absorbing moisture also absorbs heat from the patient 34. Accordingly, the absorber 26 provides a single composite material configured to generally simultaneously absorb anesthetic agent 30, heat and moisture exhaled by the patient 34. The following describes several exemplary absorber 26 compositions and methods of manufacture in detail.

According to one exemplary embodiment, the absorber 26 includes an activated carbon 26a that is comprised of an activated carbon powder, and a fibrous material 26b that is comprised of cellulose paper. The cellulose paper is saturated with water, and the activated carbon powder is applied to the cellulose paper while the cellulose paper is still wet. The moisture causes the activated carbon powder to adhere to the saturated cellulose paper. Thereafter, the absorber 26 of the present exemplary embodiment is allowed to dry such that at least a portion of the activated carbon powder remains in contact with the cellulose paper.

According to another exemplary embodiment, the absorber 26 includes an activated carbon 26a that is comprised of activated carbon fibers, and a fibrous material 26b that may, for example, comprise cellulose. The activated carbon fibers may then be interwoven with the fibrous material 26.

According to yet another exemplary embodiment, the absorber 26 includes an activated carbon 26a that is initially comprised of regular non-activated carbon fibers, and a fibrous material 26b that may, for example, comprise cellulose. The non-activated carbon fibers are interwoven with the fibrous material 26 and are thereafter activated according to a known industrial process. The industrial process by which the non-activated carbon fiber is activated is well known to those skilled in the art and will therefore not be described in detail.

According to still another exemplary embodiment, the absorber 26 includes an activated carbon 26a that is comprised of activated carbon fibers, and a fibrous material 26b that is comprised of a polymer. The activated carbon fibers can be woven together and applied to the polymer such as with adhesive thereby creating a fibrous network of both activated carbon and polymer.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A method for conserving anesthetic, heat and moisture in an anesthesia system comprising:
providing a first material configured to absorb the anesthetic transferred from a patient;
providing a second material configured to absorb the heat and moisture transferred from the patient; and
combining the first material with the second material in order to produce a single composite material adapted to generally simultaneously absorb the anesthetic, heat and moisture.

2. The method of claim 1, wherein said providing a first material includes providing an activated carbon powder.

3. The method of claim 2, wherein said providing a second material includes providing a fibrous substance.

4. The method of claim 3, wherein said providing a second material includes providing a cellulose material.

5. The method of claim 4, wherein said combining the first material with the second material includes saturating the cellulose material with water and thereafter applying the activated carbon powder to the saturated cellulose material.

6. The method of claim 1, wherein said providing a first material includes providing a plurality of carbon fibers.

7. The method of claim 6, wherein said combining the first material with the second material includes interweaving the first material with the second material.

8. The method of claim 1, wherein said providing a second material includes providing a polymer.

9. The method of claim 8, wherein said combining the first material with the second material includes adhesively applying the first material to the second material.

10. An absorber for an anesthesia system comprising:
a first material configured to absorb an anesthetic transferred from a patient; and
a second material engaged with said first material, said second material configured to absorb heat and moisture transferred from the patient;
wherein said first material and said second material are combined in a manner configured to provide generally simultaneous absorption of the anesthetic, heat and moisture.

11. The absorber of claim 10, wherein said first material includes an activated carbon powder.

12. The absorber of claim 10, wherein said first material includes a plurality of carbon fibers.

13. The absorber of claim 12, wherein said first material includes a plurality of activated carbon fibers.

14. The absorber of claim 10, wherein said second material includes a cellulose material.

15. The absorber of claim 10, wherein said second material includes a polymer.

16. An anesthesia system comprising:
a vaporizer configured to convert a liquid anesthetic agent into a gaseous anesthetic agent;
a breathing tube in communication with the vaporizer, said breathing tube configured to transfer the gaseous anesthetic agent to a patient; and
an absorber in communication with the patient, said absorber disposed remotely relative to the vaporizer, said absorber comprising:
a first material comprising carbon, said first material configured to absorb the gaseous anesthetic agent transferred from the patient; and
a second material engaged with said first material, said second material configured to absorb heat and moisture transferred from the patient;
wherein said first material and said second material are combined in a manner configured to provide generally simultaneous absorption of the anesthetic, heat and moisture.

17. The anesthesia system of claim 16, wherein said first material includes an activated carbon powder.

18. The anesthesia system of claim 16, wherein said first material includes a plurality of carbon fibers.

19. The anesthesia system of claim 18, wherein said first material includes a plurality of activated carbon fibers.

20. The anesthesia system of claim 16, wherein said second material includes a cellulose material.

21. The anesthesia system of claim 16, wherein said second material includes a polymer.
